(19) United States
(12) Patent Application Publication
(10) Pub. No.: US 2011/0110905 A1
(43) Pub. Date: May 12, 2011

(54) Infant Colic Supplement

(76) Inventor: Jennifer A. Ritchie, Laguna Niguel, CA (US)

(21) Appl. No.: 12/613,593

(22) Filed: Nov. 6, 2009

(51) Int. Cl. A61K 35/74; A61P 43/00

(52) U.S. Cl. 424/93.45

(57) ABSTRACT

Given the benefits of probiotic and digestive enzyme supplementation separately, a dietary supplement that includes both probiotic and digestive enzymes is advantageous for the treatment of infantile colic. The present invention is a novel supplement containing L. reuteri, lipase, amylase, protease, and in some formulations, lactase. The amount of each ingredient has been optimized so that it can be used, in one formulation, by a breastfeeding mother and, in another formulation, by an infant to alleviate gastrointestinal issues and in many cases decrease the incidence of colic.

### BACKGROUND

1. Field of Invention

This invention is a health supplement for infants and breastfeeding mothers consisting of a variety of ingredients to specifically reduce incidence and duration of colic through gastrointestinal regulation. Specifically, the supplement includes the bacteria L. reuteri, as well as the enzymes amylase, lipase, and protease to aid infant digestion and treat the symptoms of colic.

2. Brief Description of the Prior Art

Despite decades of research, the pathogenesis of infant colic is unclear. Infantile colic, one of the most common issues parents face in the first three months of life, is a complex behavioral syndrome characterized by inconsolable crying without identifiable cause starting at three weeks of age. Colic is diagnosed when the infant has more than three hours of crying a day, for three days a week, for more than three weeks duration. Wessel M A, et al. Paroxysmal fussing in infancy, sometimes called “colic.” Pediatrics, vol. 14 (1954), pp. 421-434. This condition can be very stressful for both mother and child. Products claiming to eliminate or reduce colic are abundant, such as simethicone drops that reduce gas. These products, however, while useful for some, do not alleviate symptoms for many infants. While historically, theories regarding the cause of infantile colic have ranged from improper maternal-infant bonding to difficult temperament, current research points to more treatable issues causing the condition.

One present theory as to the cause of the infantile colic is insufficient development of the gastrointestinal system, causing gastrointestinal distress in the infant. Recent studies supplementing infants with probiotics, specifically Lactobacillus reuteri, have shown a decrease in colic. Savino et al. Lactobacillus reuteri (American type Culture Collection Strain 55730) Versus Simethicone in Treatment of Infantile Colic: A Prospective Randomized Study. Pediatrics; 2007: 119: e124-130. This study used L. reuteri as it is one of the few endogenous Lactobacillus species in the human gastrointestinal tract. It has been used for many years as a probiotic dietary supplement and has been shown to be safe even after long-term supplementation in infants. Connolly et al. Safety of D(−) lactic acid producing bacteria in the human infant. Journal of Pediatric Gastroenterology and Nutrition. 2005; 41: 489-492. Probiotics like L. reuteri, by definition benefit the consumer by improving intestinal microbial balance.

Insufficient digestive enzymes affect adult and infant digestion as well. This deficiency is also posited to be the cause of some infantile colic. Insufficient digestive enzymes can be particularly deleterious to the breastfeeding mother, who is passing those enzymes on to the infant. If the mother is deficient in these enzymes, then the infant will necessarily be deficient. It is known that adults who consume large amounts of foods devoid of enzymes results in a depletion of enzymes in that adult's body. This puts a strain on the pancreas to secrete greater amounts of digestive enzymes than normal, exhausting the pancreas. In response, the immune system increases its production of white blood cells that have stores of enzymes to aid in the digestive process. As a result, the adult suffers impaired immune function.

Adults have the ability to eat certain foods to avoid enzyme depletion. These foods include raw meat, unpasteurized milk, fresh fruits and vegetables, fermented foods, sprouted seeds, and nuts that have been soaked in water. Unfortunately, not all of these foods are available or safe for consumption. Breast milk is another natural source rich in enzymes. Breast milk, because it is not pasteurized or cooked, contains natural enzymes that aid in digestion and require the infant consumer little effort to digest. Cow's milk and infant formulas are heat-treated through pasteurization, a process that results in killing the natural digestive enzymes. Because an infant consuming these products cannot digest them fully, partially digested protein particles will pass through the intestinal lining, and may cause an allergic response. The immune system will resultantly be depleted of enzymes, and not function optimally. Constant exposure to proteins that are not fully digested have been hypothesized to cause a variety of maladies including: arthritis, Candida, environmental allergies and sensitivities, asthma, headaches, fatigue, muscle pain, heart disease, obesity, eczema, and hyperactivity. Extra digestive enzymes can be given to prevent this dilemma.

May infant formulas now contain some form of digestive enzymes, although it has been posited that such enzymes are not as effective as those in breast milk. Because the majority of infants are supplemented by infant formula, it would be advantageous to have a supplement that is bio-accessible by infants, and can be used by breastfeeding mothers as well, to be passed on to the infant.

Another theory regarding the genesis of infantile colic includes the infant being allergic to components of the breast milk, which in some cases may be alleviated by modifying the maternal diet. Hill et al. A low allergen diet is a significant intervention in infantile colic: results of a community-based study. Journal of Allergy and Clinical Immunology. 1995; 96: 886-892. Because proteins pass from the mother to infant through breast milk, some of these intact proteins may cause allergic response and colic symptoms. Pittschieler. Cow's milk protein-induced colitis in the breastfed infant. Journal of Pediatrics Gastroenterology and Nutrition. 1990; 10: 548-549. Supplementing the breastfeeding mother with digestive enzymes, specifically PANCREASE, has also been shown to have a positive effect on infants suffering from colic who are nursing. Schack and Haight. Colic and Food Allergy in the Breastfed Infant: Is it Possible for an Exclusively Breastfed Infant to Suffer from Food Allergy? Journal of Human Lactation; 2002: 18(1): 50-52.

Presently, no supplement exists that addresses the benefits of probiotics and digestive enzymes in one supplement for infants suffering from colic or other gastrointestinal issues.

### SUMMARY OF THE INVENTION

Given the benefits of probiotic and digestive enzyme supplementation separately, a dietary supplement that includes both probiotic and digestive enzymes is advantageous for the treatment of infantile colic. The present invention is a novel supplement containing L. reuteri, lipase, amylase, protease, and in some formulations, lactase. The amount of each ingredient has been optimized so that it can be used, in one formulation, by a breastfeeding mother and, in another formulation, by an infant to alleviate gastrointestinal issues and in many cases decrease the incidence of colic.

It is an object of the present invention to provide a supplement to infants and breastfeeding mothers to aid in digestion.

It is another object of the present invention to provide a supplement for infants and breastfeeding mothers to reduce episodes of colic.

It is a further object of the present invention to provide a supplement for infants and breastfeeding mothers to increase overall health and well-being.

It is an additional object of the present invention to provide a supplement that is easy to administer to infants and breastfeeding mothers.

It is another object of the present invention to provide a supplement that is cost effective and readily available to the caregivers of an infant and breastfeeding mothers.

Still other aspects of the present invention will become apparent to those skilled in the art from the following description of a preferred embodiment, which is by way of illustration, one of the best modes contemplated for carrying out the invention. As will be realized, the invention is capable of other different and obvious aspects, all without departing from the invention. Accordingly, the descriptions are illustrative in nature and not restrictive.

### DETAILED DESCRIPTION

A detailed explanation of the present invention is described below. The described preferred embodiments are presented for the purpose of illustration and description; they are not intended to limit the scope of the present invention. As will be apparent to one skilled in the art, modifications to described elements below may be made without deviating from the scope of the present invention.

The present invention provides a supplement that supports healthy digestion in infants. The supplement may also be taken by breastfeeding mothers to aid their infant's digestion through their breast milk. Use of the supplement may, in many cases, provide relief of infantile colic symptoms, as in one preferred embodiment of the invention. The supplement is composed of: L. reuteri, lipase, amylase, and protease. The infant supplement is composed of: L. reuteri, and lactase, but may contain lipase, amylase, and protease. Other species of Lactobacillus that aid in digestion and immune function may be used as part of the supplement.

L. reuteri is a gram-positive bacterium from the Lactobacillus species that naturally inhabits the gut of mammals and birds. It is named for the discoverer Gerhard Reuter. The bacteria has been used to treat rotavirus-induced diarrhea in children, is effective for the prevention of various gut infections, capable of promoting dental health, and ma protect against common general infections. Because of the history of treatment with the bacteria in children, it is considered very safe to use, even in infants.

Lipase is a water-soluble enzyme that catalyzes the hydrolysis of ester bonds in water-insoluble, lipid substrates. Lipases are essential to healthy digestion and in the transport and processing of dietary lipids (fats and oils) in most organisms. The main lipase of the human digestive system is human pancreatic lipase (HPL) that is secreted by the pancreas.

Lactase is an enzyme located in the small intestine that digests the sugar found in milk and milk products, lactose.

Amylase is an enzyme present in saliva that breaks down starches and sugars, beginning the process of digestion. Amylase is made in the pancreas to hydrolyze dietary starch into di- and trisaccharides, which is eventually converted to glucose in conjunction with other enzymes.

Protease is a class of enzymes made in the pancreas that digest proteins. Protease works by conducting proteolysis that begins protein catabolism by the hydrolysis of peptide bonds that create proteins. Two specific examples of proteases suitable for human consumption include trypsin and chymotrypsin.

A USP unit is a dose unit as recommended by the United States Pharmacopoeia, the primary legally recognized national drug-standard compendium, and it expresses the potency of drugs and other preparations.

A CFU is a “Colony-forming unit”, which is the minimum number of cells on the surface or in a semi-solid agar medium which gives rise to a visible colony of progeny is on the order of tens of millions. CFUs may consist of pairs, chains, and clusters as well as single cells and are often expressed as colony-forming units per milliliter (CFU/ml).

In one preferred embodiment of the invention the supplement is taken by an infant orally, one time per day. The form for the infant formulation is in a liquid, so that is easy for a caregiver to give the infant. Dosage for the infant formulation is slightly different than that for the adult, as in one embodiment. For example, the infant dosage is 100 Million CFU of L. reuteri and 432 lactase units of lactase enzyme. In another embodiment, the infant supplement includes L. reuteri, protease (trypsin and chymotrypsin), amylase, lipase and/or lactase. Other species of Lactobacillus are contemplated for use as part of the present invention. Other ingredients may be added to the suspension to aid in taste, texture, and volume, as is apparent to those skilled in the art. As in one embodiment, the suspension contains glycerol purified water, sodium chloride, sunflower oil, medium chain triglyceride and silicone dioxide. Proportions and volume of each inactive ingredient may be optimized for taste and texture, as is known to those skilled in the art. Other suitable inactive ingredients are known to those skilled in the art and may be employed without deviation from the present invention.

In another preferred embodiment of the invention, the supplement is taken by a breastfeeding mother, or adult, about 2 times daily in oral form. This oral form may be either a pill or a chewable, as would be apparent to one skilled in the art. The pill-form of the present invention contains approximately 100 million CFU of active L. reuteri Protectis bacteria; 50,000 USP units of amylase; 50,000 USP units of protease (trypsin and chymotrypsin); 9,000 USP units of lipase, as in the preferred embodiment of the present invention, per dose (2 pills are taken daily). In another embodiment of the invention, lactase enzyme may also be present. Other species of Lactobacillus are contemplated for use as part of the present invention. The pill-form of the present invention may also contain fillers so that the pill is of a suitable size for consumption. Fillers that may be employed include, but are not limited to: rice flour and magnesium stearate, as in the preferred embodiment of the present invention. Other fillers may be employed, as is known by those skilled in the art. When the pill form is ingested by a breastfeeding mother, the benefits of the bacteria, and broken down proteins (as a result from the enzymes) are passed on to the nursing infant or child.

I claim:

1. A digestive supplement comprising Lactobacillus, amylase, protease (trypsin and chymotrypsin), lipase, and/or lactase in an ingestible carrier.

2. A digestive supplement comprising the recited constituents of approximately 100 million active Lactobacillus reuteri, approximately 100,000 USP units of amylase, approximately 100,000 USP units of protease (trypsin and chymotrypsin), and approximately 18,000 USP units of lipase.

3. The digestive supplement of claim 2, wherein each of said recited constituents is divided between two pharmaceutically acceptable carriers that may be taken orally.

4. The digestive supplement of claim 2, wherein said ingestible carrier is a pill form, wherein the pill may contain rice flour and/or magnesium stearate.

5. The digestive supplement of claim 2, wherein the recited constituents when administered to a breastfeeding mother, may alleviate symptoms of infantile colic in a nursing infant.

6. A digestive supplement for infants comprising Lactobacillus reuteri and lactase enzyme.

7. A digestive supplement for infants comprising 100 million CFU of Lactobacillus reuteri, 432 lactase units of lactase enzyme and inert ingredients including glycerol purified water, sodium chloride, sunflower oil, medium chain triglyceride and silicone dioxide in a suspension.

8. The digestive supplement of claim 7, wherein the suspension is delivered twice daily to the infant.

9. The digestive supplement of claim 7, wherein the recited constituents may alleviate the symptoms of infantile colic when administered to the infant daily.

10. A digestive supplement comprising the recited constituents of approximately 100 million active Lactobacillus reuteri, approximately 100,000 USP units of amylase, approximately 100,000 USP units of protease (trypsin and chymotrypsin), and approximately 18,000 USP units of lipase, wherein said recited constituents are delivered in pill form; said pill containing rice flour and/or magnesium stearate and said pill containing approximately one half of the said recited constituents so that a user may take two pills per day to consume the doses recited for each of said constituents.

11. The digestive supplement of claim 11, wherein the recited constituents when administered to a breastfeeding mother, may alleviate symptoms of infantile colic in a nursing infant.
